(19) United States
(12) Patent Application Publication
(10) Pub. No.: US 2015/0191445 A1
(43) Pub. Date: Jul. 9, 2015

(54) ACYL-HYDRAZONE AND OXADIAZOLE COMPOUNDS, PHARMACEUTICAL COMPOSITIONS CONTAINING THE SAME AND USES THEREOF

(71) Applicant: Universidade Federal de Santa Catarina, Florianopolis (BR)

(72) Inventors: Ricardo Jose Nunes, Florianopolis (BR); Alessandra Mascarello, Florianopolis (BR); Rosendo Augusto Yunes, Florianopolis (BR); Taisa Regina Stumpf, Florianopolis (BR); Paulo Cesar Leal, Palhoca (BR); Jose Andres Yunes, Campinas (BR); Carolina Pereira de Souza Melo, Itabirito (BR); Rafael Renatino Canevarolo, Campinas (BR); Louise Domeneghini Chiaradia, Florianopolis (BR); Andre Bortolini Silveira, Campinas (BR); Angelo Brunelli Albertoni Laranjeira, Campinas (BR)

(21) Appl. No.: 14/360,279

(22) PCT Filed: Nov. 26, 2012
(86) PCT No.: PCT/BR2012/000480

§ 371 Date: Mar. 19, 2015

(30) Foreign Application Priority Data
Nov. 25, 2011 (BR) PI 1107312-8

(51) Int. Cl. C07D 333/22; C07D 233/64; C07C 243/38

(52) U.S. Cl. CPC C07D 333/22; C07C 243/38; C07D 233/64

(57) ABSTRACT

The present invention relates to acyl-hydrazone compounds, in particular 3,4,5-trimethoxyphenyl-hydrazide derivatives, as well as the oxadiazole analogs thereof and other similar compounds, and to the pharmaceutical use of the same for the treatment of various diseases associated with cell proliferation, such as leukemias, including acute lymphoblastic leukemia (ALL), tumours and inflammation. Acyl-hydrazones have been obtained having activity similar to that of the compound used as a standard in experiments (colchicine). The greater selectivity of the compounds according to the invention is an important feature, associated with fewer side effects than the pharmaceuticals used at present in clinical treatments. The synthetised acyl-hydrazones, more particularly the compounds 02 and 07, exhibited important antileukemic activity, which suggests 02 and 07 as candidates to pharmaceutical prototypes, or to pharmaceuticals for the treatment of leukemias, in particular acute lymphoblastic leukemia (ALL), tumours and other proliferative diseases, such as inflammation. The action mechanism of the most active compounds was determined by using DNA microarrays and subsequent tests indicated by the chip, besides selectivity studies in healthy human lymphocytes.

### BACKGROUND

1. Technical Field

The present invention relates to acyl-hydrazones derived from 3,4,5-trimethoxyphenyl hydrazide for the treatment of diseases associated with cell proliferation (such as leukemias, tumors, inflammation and other proliferative diseases). More specifically, the invention relates to compounds having inhibitory activity against cyclin dependent protein kinases (CDKs) and topoisomerase I, which may therefore be useful in treating acute lymphoblastic leukemia (ALL). The invention further relates to obtaining novel acyl-hydrazones and oxadiazoles from 3,4,5-trimethoxyphenyl-hydrazide.

2. Description of the Related Art

Cancer is a disease characterized by proliferation and uncontrolled spread throughout the body of abnormal forms of human cells. The neoplastic cells differ from normal cells by the high invading capability they possess, by the loss of function, by the loss of differentiation and by the ability to metastasize, by virtue of having lower adherence among themselves (Rang, H. P.; Dale, M. M.; Ritter, J. M; Moore, P. K. Farmacologia. 5 ed. Rio de Janeiro: Elsevier, 2004. 703 p). In 2009, it was estimated the occurrence of 49,000 new cases of breast cancer, 47,000 of prostate cancer, 27,000 of lung cancer, 25,000 of colon and rectum cancer, 23,000 of stomach cancer and 19,000 of cervical cancer in Brazil in 2009 (INCA, Instituto Nacional do Cancer [National Cancer Institute]. Available at: <http://www.inca.gov.br/estimativa/2006/index.asp?link=mapa.asp&ID=8> Accessed on: Feb. 23, 2011), being the second leading cause of death in the country.

Leukemia is one of several types of cancer, and occurs by neoplastic proliferation of lymphoid or myeloid hematopoietic cells, resulting from the mutation of a single stem cell, whose progeny form a clone of leukemic cells. Multiple genetic alterations for malignant transformation usually occur, including inappropriate expression of oncogenes and loss of function of tumor suppressor genes (Bain, B. J. Diagnóstico em leucemias. Rio de Janeiro: Elsevier, 2003, Cap. 1, 01-56), that may be associated with genetic factors or risks (such as smoking, exposure to radiation or chemicals such as benzene) (INCA, Instituto Nacional do Câncer [National Cancer Institute]—Leucemia—prevenção, genética, outros fatores de risco [Leukemia—prevention, genetics, other risk factors] available at: <http://www2.inca.gov.br/wps/wcm/connect/tiposdecancer/site/home/leucemia/pre vencao_genetica_outros_fatores_de_risco>. Accessed on: Feb. 23, 2011; and IARC (International Agency for Research on Cancer). World Cancer Report 2008. Available at: <http://www.iarc.fr/en/publications/pdfs-online/wcr/2008/index.php> and <http://www.iarc.fr/en/publications/pdfs-online/wcr/2008/wcr2008.pdf>. Accessed on: Feb. 23, 2011). Leukemias are further subdivided based on how quickly the disease progresses and becomes severe, and may be acute or chronic. Acute leukemias are characterized by a defect in cell maturation, which leads to an imbalance between proliferation and maturation; since the leukemic clone of cells continue to proliferate without reaching the stage of maturation and death, leading to a continued expansion of the leukemic clone and predominance of immature cells (INCA 2009b, Bain, B. J. Diagnóstico em leucemias. Rio de Janeiro: Elsevier, 2003, Cap. 1, 01-56).

Acute lymphoblastic leukemia (ALL) is due to the uncontrolled proliferation of immature lymphoid progenitor cells in the bone marrow, resulting in a very rapid accumulation of neoplastic cells (Plasschaert, S.; Van Der Kolk, D.; De Bont, E.; Vellenga, E.; Kamps, W.; De Vries, E. Breast Cancer Resistance Protein (BCRP) in Acute Leukemia. Leukemia & Lymphoma, 2004, 45, 649-654). It accounts for 80% of the cases of acute leukemia in childhood (Laks, D.; Longhi, F., Bernardes, W. M.; Ramos, G. P. C. J Pediatr, 2003, 79, 149-158) and for 50% of all hematopoietic malignancies (Downing, James R.; Shannon, Kevin M. Acute leukemia: A pediatric perspective. Cancer Cell, 2002, 2, 437-445). In adults, ALL is relatively rare, accounting for 2-3% of hematopoietic malignancies (Downing, James R.; Shannon, Kevin M. Acute leukemia: A pediatric perspective. Cancer Cell, 2002, 2, 437-445); however, the prognosis is much worse than for children because it affects multipotent stem cells, yielding a much more aggressive leukemia (Greaves, M. F. Stem cell origins of leukaemia and curability. British Journal of Cancer, 1993, 67, 413-423).

The research on the use of compounds from natural sources as chemotherapy is ample. An example is the antineoplastic paclitaxel (Taxol™) one of the most important antitumor natural products ever discovered, first reported in 1971 (Wani, M. C.; Taylor, H. L.; Wall, M. E.; Coggon, P.; McPhail, A. T. Plant antitumor agents. VI. Isolation and structure of taxol, a novel antileukemic and antitumor agent from Taxus brevifolia. Journal of the American Chemical Society, 1971, 93, 2325-2327), that only after a long period has been approved by the FDA for clinical use (in 1992). Another example is vincristine, an alkaloid used in the therapy for acute leukemia and other types of tumors, which operates the same way as paclitaxel by binding to tubulin, interfering with the formation (polymerization) or reorganization (depolymerization) of microtubules (Goodman e Gilman. As bases farmacológicas da terapêutica, 10^{a} Ed., editora Mc Graw Hill. 2006).

The most commonly used chemotherapeutic agents in the current therapy for leukemia include daunorubicin, doxorubicin, dexamethasone, vincristine, methotrexate and mercaptopurine (Plasschaert, S.; Van Der Kolk, D.; De Bont, E.; Vellenga, E.; Kamps, W.; De Vries, E. Breast Cancer Resistance Protein (BCRP) in Acute Leukemia. Leukemia & Lymphoma, 2004, 45, 649-654). These drugs provide therapeutic benefit, but also significant toxicity to the body and normal cells, due to their role in the induction of apoptosis and inhibition of cell proliferation (Herr, I.; Debatin, K. M. Cellular stress response and apoptosis in cancer therapy. Blood 2001, 98, 2603-2614; and Leszczyniecka, M.; Roberts, T.; Dent, P.; Grant, S.; Fisher, P. B. Differentiation therapy of human cancer: basic science and clinical applications. Pharmacology & Therapeutics, 2001, 90, 105-156). Antineoplastic agents also interfere with normal tissues that have rapidly dividing cells, which can cause many undesirable effects, such as reducing the production of defense cells in the body, poor wound healing, alopecia, gastrointestinal epithelial injury, sterility and teratogenicity (Rang, H. P.; Dale, M. M.; Ritter, J. M; Moore, P. K. Farmacologia. 5 ed. Rio de Janeiro: Elsevier, 2004. 703 p).

Despite recent advances in cancer research, in 2008 there were 5,686 registered deaths from leukemia in Brazil, and for 2010 there were an estimated 9,580 new cases of the disease (INCA, Instituto Nacional do Cancer [National Cancer Institute]. Available at: <http://www.inca.gov.briestimativa/2006/index.asp?link=mapa.asp&ID=8> Accessed on: Feb. 23, 2011), which constantly motivates the search for new drugs for the treatment of leukemia and other tumors.

In a recent study, Wang and colleagues (Wang, Z.; Lu, Y.; Seibel, W.; Miller, D. D.; Li, W. Identifying Novel Molecular Structures for Advanced Melanoma by Ligand-Based Virtual Screening. Journal of Chemical Information and Modeling, 2009, 49, 1420-1427) evaluated new antitumoral compounds based on the structure of the compound LY-1-100, which has a proven mechanism of action and affinity for microtubules (colchicine binding site), but low selectivity.

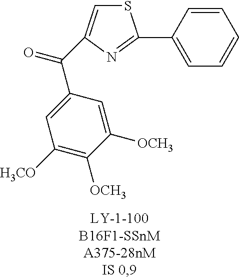 [SMILES: COc1cc(C(=O)c2csc(-c3ccccc3)n2)cc(OC)c1OC]

Structures were selected by ligand-based virtual screening, resulting in 14 new molecules with high structural similarity to the starting compound (LY-1-100). The theoretical results obtained by researchers revealed that the trimethoxylated ring of the LY-1-100 compound must be maintained to provide antitumor activity, but that the triazole ring doesn't seem to be important and could be replaced by the structure of N-methylene-hydrazine, which generates the structure of acyl-hydrazones. Although the results in murine B16F1 and A375 cells (in vitro) of these compounds have not shown improved potency and selectivity on the starting compound, these results were very promising and identified some major structural components for anti-melanoma activity (Wang, Z.; Lu, Y.; Seibel, W.; Miller, D. D.; Li, W. Identifying Novel Molecular Structures for Advanced Melanoma by Ligand-Based Virtual Screening. Journal of Chemical Information and Modeling, 2009, 49, 1420-1427).

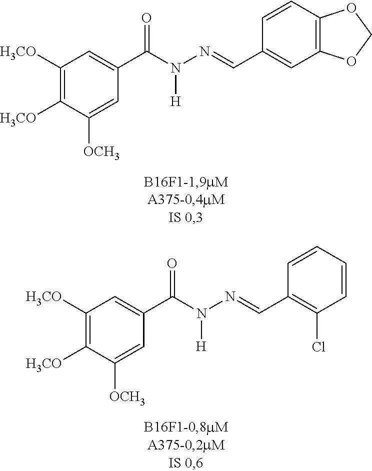 [SMILES: COc1cc(C(=O)N/N=C/c2ccc3c(c2)OCO3)cc(OC)c1OC ; COc1cc(C(=O)N/N=C/c2ccccc2Cl)cc(OC)c1OC]

In this regard, acyl-hydrazones appear as an interesting class of compounds with antitumor activity. Thus, the present invention relates to obtaining acyl-hydrazones, especially those derived from 3,4,5-trimethoxyphenyl-hydrazide for the treatment of diseases associated with cell proliferation (such as leukemias, tumors, inflammation and other proliferative diseases).

The patent application PI 0112674-1 (n-[5-[[[5-alkyl-2-oxazolyl]methyl]thio]-2-tiazolyl]carboxamide inhibitors of cyclin-dependent kinases) describes compounds and their enantiomorphs, diastereomers, solvates and pharmaceutically acceptable salts thereof as inhibitors of protein kinases useful in the treatment of proliferative diseases such as cancer, inflammation and arthritis.

The synthesized compounds can also be useful for the treatment of Alzheimer's disease, chemotherapy-induced alopecia and cardiovascular disease. The compounds described in the application PI 0112674-1 represent more complex structures than those presented in the course of the present invention.

The compounds of the referenced patent application publication No. U.S. 20040138272 (1,4-Substituted cyclohexane derivatives) may be useful in preventing cell proliferation in malignant diseases by inhibiting Rho kinases, useful in the repair of central and peripheral nervous system by growth induction and regeneration of axons. The mechanism of action of the compounds of publication U.S. 20040138272 differs from that proposed for the structures of the present invention.

Inhibitors of cyclin-dependent kinases (cdks) useful in modulating cell-cycle progression are proposed in patent application PI 0418095-0 (inhibitor of cyclin-dependent kinases, compositions and uses related thereto). Such compounds would be useful for the treatment of patients with disorders associated with excessive cell proliferation. The compounds described in PI 0418095-0 are acyl-hydrazones different from those proposed in the course of the present invention with a more complex synthesis process.

In the patent application PI 0508364-8 (derivatives of 4-benzimidazol-2-yl-pyridazin-3-one) compounds are described and physiologically tolerated salts thereof, which have activity as inhibitors of kinases, in particular CDK2 kinase (cyclin-dependent kinase 2). The compounds of PI 0508364-8 are different from the proposed in the course of the present invention with a more complex synthesis.

The publication of U.S. patent application No. U.S. 20070066610 (Acyl-hydrazones as kinase modulators) discloses acyl-hydrazones as inhibitors of tyrosine kinases, comprising c-Met, a tyrosine kinase receptor which regulates cellular proliferation, morphogenesis, and motility. The acyl-hydrazones described in U.S. 20070066610 are different from those proposed in the course of this invention, with a more complex synthesis. In addition, the target of action of the compounds described differs from the proposed herein.

The published U.S. patent application 20080194562 (Pyrazole Derivatives for The Inhibition of Cdk's And Gsk's) refers to the synthesis of pyrazole compounds that inhibit or modulate the activity of cyclin-dependent kinases (CDK) and kinase glycogen synthase (GSK), and their use in the treatment or prophylaxis of kinase mediated diseases or conditions. Pharmaceutical compositions containing the compounds and chemical intermediates are also described. The compounds of the U.S. 20080194562 document are different from the proposed in the course of the present invention.

Acyl-hydrazones are also described in the literature for their pronounced insecticidal and plant growth stimulation activities (Robinson, B. Fischer indole synthesis. Chem. Rev., 1963, 4, 373-401); for the treatment of tuberculosis (Vigorita, M. G.; Ottana, R.; Zappala, C.; Maccari, R.; Pizzimenti, F. C.; Gabbrielli, G. Halogenated isoniazid derivatives as possible antimycobacterial and anti-HIV agents—III. Farmaco, 1994, 49, 775-781); and as bacteriological and bacteriostatic agents (Samus, N. M.; Tsapkov, V. I.; Kuracheva, S. A.; Burdenko, T. A. Synthesis and antimicrobial activity of coordination compounds of 3d-elements with some hydrazones derived by using 5-nitro-2-furaldehyde. Khimiko-Farmatsevticheskii Zhurnal, 1994, 28, 41-44).

Considering what has been stated above, a study was conducted with acyl-hydrazones and their oxadiazole derivatives obtained by the non-classic bioisosterism strategy of ring closure, with the goal of developing new chemotherapeutic agents. The oxadiazoles are an important class of heterocyclic compounds with a wide range of biological activities, such as antiviral, antimicrobial, antineoplastic, fungicidal, and inhibition of tyrosinase and cathepsin K (Kumar, D.; Sundaree, S.; Johnson, E. O.; Shah, K. An efficient synthesis and biological study of novel indolyl-1,3,4-oxadiazoles as potent anticancer agents. Bioorganic & Medicinal Chemistry Letters, 2009, 19, 4492-4494). Also, they are great bioisosters of amides and esters, which can substantially contribute to increased pharmacological activity, participating in hydrogen bonds as receptors (Guimaraes, C. R. W.; Boger, D. L.; Jorgensen, W. L. Elucidation of Fatty Acid Amide Hydrolase Inhibition by Potent α-Ketoheterocycle Derivatives from Monte Carlo Simulations. Journal of the American Chemical Society, 2005, 127, 17377-17384).

Thus, the specific acyl-hydrazones and their analogues, from the processes from this invention described in detail below, as well as their use in the treatment of leukemia, tumors and other proliferative diseases such as inflammation, is of great economic and social interest.

### BRIEF SUMMARY

The aim of the present invention is to obtain synthetic compounds derived from 3,4,5-trimethoxyphenyl-hydrazide (hydrazones and oxadiazoles) and all analogs, and the like, by means of a simple synthetic route, as well as the use of these compounds for the treatment of diseases associated with cell proliferation (such as leukemias, especially acute lymphoblastic leukemia—ALL—tumors, inflammation and other proliferative diseases). The present invention also describes procedures used to determine the biological activity of these compounds.

The present invention relates to a class of acyl-hydrazones, especially those derived from 3,4,5-trimethoxyphenyl-hydrazide, as well the analog compounds oxadiazole and other similar and related compounds, and the pharmaceutical application of all these in the treatment of different diseases associated with cell proliferation such as leukemia, including acute lymphoblastic leukemia (ALL), tumors and inflammation. The present invention also describes procedures used for the determination of biological activity of all these compounds.

Acyl-hydrazones with a similar activity to the compound used as a standard in the experiments (colchicine) were obtained. The higher selectivity of the compounds disclosed herein is an important feature related with fewer side effects than the drugs currently used in the clinic practice. The acyl-hydrazones synthesized, more specifically the compounds 02 and 07 showed significant antileukemic activity, which indicates 02 and 07 as candidates for drugs prototypes or drugs, for the treatment of leukemias, especially acute lymphoblastic leukemia (ALL), tumors and other proliferative diseases such as inflammation.

The determination of the action mechanism of the most active compounds was performed by using DNA microarrays and subsequent tests indicated through the chip, in addition to selectivity studies in healthy human lymphocytes.

### DESCRIPTION OF THE SEVERAL VIEWS OF THE DRAWINGS

FIG. 1 shows (A) Overview of the enrichment of the gene set defined as “REACTOME POST CHAPERONIN TUBULIN FOLDING PATHWAY” showing enrichment in control samples HL-60 versus samples treated with compound 07 (p=0.002, FDR q-value=0.168) and (B) The expression values of the 16 genes that are part of the set of genes in (A) are represented as blue squares (lower expression) and red squares (higher expression).

FIG. 2 shows that Compound 07 induces cell cycle arrest in G2/M. Jurkat cells treated for 18 h with 125 nM of compound 07 or DMSO were subjected to cell cycle analysis after staining with propidium iodide and flow cytometry analysis.

FIG. 3 shows the results of Western blot analysis for various cell cycle regulators protein extracts from Jurkat cells treated with 125 nM of compound 07 (F8) or vehicle (DMSO).

FIG. 4 shows that compound 07 is a strong inducer of apoptosis in Jurkat cells. Overall, the results suggest that compound 07 promotes cell cycle arrest and apoptosis, mainly through Chk2 and Rb. Jurkat cells treated for 18 h with 125 nM of compound 07 or DMSO were double stained with annexin V/propidium iodide and analyzed by flow cytometry.

FIG. 5 shows the effect of compound 07 on human lymphocytes (WBC) and leukemic Jurkat and HEK cells after 48 h. The proliferation of normal lymphocytes were stimulated with phytohemagglutinin. The percentage of the survival of cells treated with compound 07 versus the survival of cells treated with vehicle (DMSO) is shown.

### DETAILED DESCRIPTION

The present invention comprises obtaining and the action mechanism of synthetic acyl-hydrazones and its analog and related compounds, which may be useful in treating leukemias, especially acute lymphoblastic leukemia (ALL), tumors and other proliferative disorders such as inflammation.

According to one of the aspects of the present invention, a structure compound of (I) is described:

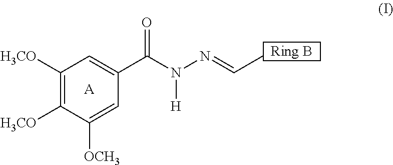

wherein Ring B represents:

01 - phenyl
02 - 4-Br-phenyl
03 - 4-NO₂-phenyl
04 - 3-OCH₃-4-OH-5-Br-phenyl
05 - 3-OCH₃-4-OH-5-I-phenyl
06 - 3-OCH₃-4-OH-phenyl
07 - 1-naphthyl
08 - 2-naphthyl
09 - 4-Cl-phenyl
10 - 3,4-OCH₂O-phenyl
11 - 2-Cl-phenyl
12 - 2,5-diOCH₃-phenyl
13 - 3,4,5-triOCH₃-phenyl
14 - 2,4,5-triOCH₃-phenyl
15 - 4-O(CH₂)₃CH₃-phenyl
16 - 4-CH3-phenyl
17 - 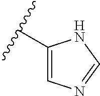 [SMILES: *c1cnc[nH]1]
18 - 3-NO₂-phenyl
19 - 4-N(CH₃)₂-phenyl
20 - 3-Cl-phenyl
21 - 2,6-diOCH₃-phenyl
22 - 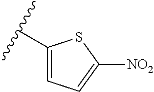 [SMILES: *c1ccc([N+](=O)[O-])s1]
23 - 2-COOH-phenyl
24 - 3-OCH₂CH₃-4-OH-phenyl
25 - 3-OCH₃-phenyl
26 - 2-OH-4-Br-phenyl
27 - (3-OCH₃-4-OCH₂phenyl)-phenyl
28 - 4-CF₃-phenyl
29 - 3-CF₃-4-Cl-phenyl

A further group of compounds according to the present invention comprises compounds having the structure II:

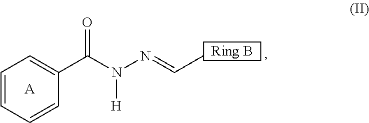

wherein Ring B represents:

30—phenyl

31—3,4-OCH₂O-phenyl

32—4-Br-phenyl

33—4-CH₃-phenyl

34—1-naphthyl

35—2-naphthyl

Additionally, synthetic analogs oxadiazoles are also described, according to the structure III:

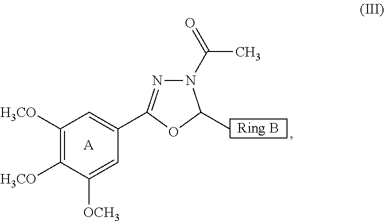

wherein ring B represents:

36—3-OCH₃-4-OH-5-Br-phenyl

37—3-OCH₃-4-OH-phenyl

38—1-naphthyl

The novel acyl-hydrazones of the present invention were obtained from the condensation reaction between 3,4,5-trimethoxyphenyl-hydrazide and different aldehydes using ethanol as solvent at reflux, according to the reaction:

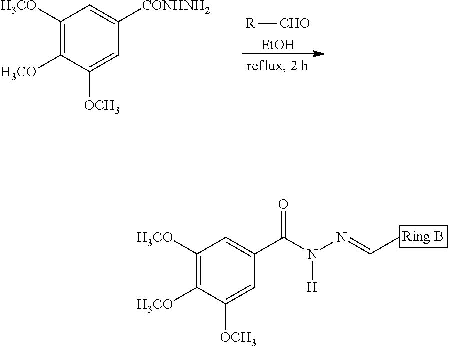

wherein ring B represents:

02 - 4-Br-phenyl*
05 - 3-OCH₃-4-OH-5-I-phenyl
07 - 1-naphthyl*
17 - 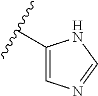 [SMILES: *c1cnc[nH]1]
21 - 2,6-diOCH₃-phenyl
26 - 2-OH-4-Br-phenyl
29 - 3-CF₃-4-Cl-phenyl

Table 1 shows the yields obtained in the synthesis and the experimental melting points of the novel 3,4,5-trimethoxyphenyl-hydrazones.

TABLE 1
Yields and melting points of novel synthesized 3,4,5-
trimethoxyphenyl-hydrazones.

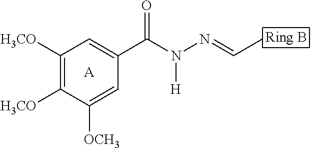

| | | | Experimental |
|---|---|---|---|
| | | Yield | melting |
| Compound nr. | Ring B | (%) | point (° C.) |
| 02* | 4-Br-phenyl | 82 | 208-209 |
| 05 | 3-OCH₃-4-OH-5-I-phenyl | 69 | 247-248 |
| 07* | 1-naphtyl | 82 | 229-230 |
| 17 | 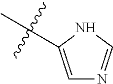 [SMILES: *c1cnc[nH]1] | 75 | 230-231 |
| 21 | 2,6-diOCH₃-phenyl | 93 | 245-246 |
| 26 | 2-OH-4-Br-phenyl | 65 | 206-208 |
| 29 | 3-CF₃-4-Cl-phenyl | 83 | 203-204 |

The structure of compound 02 was previously published as the reaction intermediate for obtaining oxadiazole (Mazzone, G.; Bonina, F.; Formica, F. Some aroylhydrazones of halobenzaldehydes and halo-substituted 2,5-diaryl-1,3,4-oxadiazoles. Farmaco, Edizione Scientifica, 1978, 33(12), 963-71) and compound 07 was previously evaluated as a MAO (monoamine oxidase) inhibitor (Mazzone, G.; Arrigo Reina, R. 3,4,5-Trimethoxybenzoyl hydrazides and their anti-MAO [monoamine oxidase] activity. Bollettino delle Sedute della Accademia Gioenia di Scienze Naturali in Catania, 1971, 10(8), 689-702). The chemical characterization of both compounds (02 and 07) was also previously published by our research group, in a study that evaluated the activity of these and other compounds as inhibitors of Trypanosoma cruzi cruzain, however, the compounds 02 and 07 did not show inhibitory activity of this protein (Borchhardt, Deise M.; Mascarello, Alessandra; Chiaradia, Louise Domeneghini; Nunes, Ricardo J.; Oliva, Glaucius; Yunes, Rosendo A.; Andricopulo, Adriano D. Biochemical evaluation of a series of synthetic chalcone and hydrazide derivatives as novel inhibitors of cruzain from Trypanosoma cruzi. Journal of the Brazilian Chemical Society, 2010, 21(1), 142-150). However, the use of the compounds 02 and 07 for the treatment of leukemia is not described in the prior art and is within the scope of the invention.

The additional acyl-hydrazones (prepared to assist in the discussion of biological tests) were synthesized by the reaction between the hydrazide and phenyl different aldehydes by the same procedure for the preparation of hydrazides described above, according to the following reaction:

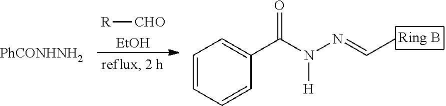

wherein Ring B represents:

30—phenyl

31—3,4-OCH₂O-phenyl

32—4-Br-phenyl

33—4-CH₃-phenyl

34—1-naphthyl

35—2-naphthyl

Novel synthetic analogs oxadiazoles of 3,4,5-trimethoxyphenyl-hydrazones were also prepared, from the reaction of these with acetic anhydride according to the following reaction:

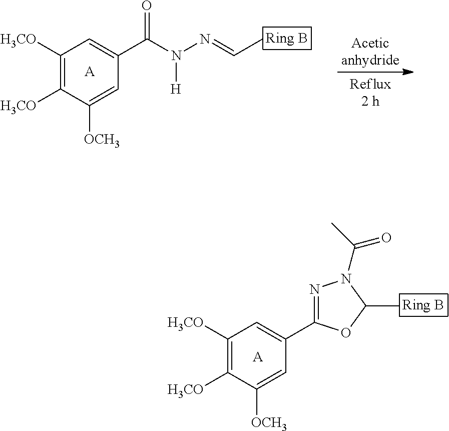

wherein ring B represents:

36—3-OCH₃-4-OH-5-Br-phenyl

37—3-OCH₃-4-OH-phenyl

38—1-naphthyl

Table 2 shows the yields obtained in the synthesis and experimental melting point of the oxadiazoles.

TABLE 2
Yields and melting point of the novel synthesized oxadiazoles.

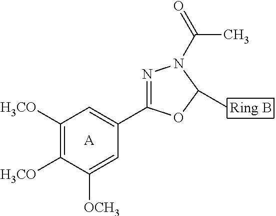

| | | | Experimental |
|---|---|---|---|
| | | Yield | melting |
| Compound nr. | Ring B | (%) | point (° C.) |
| 36 | 3-OCH₃-4-OH-5-Br-phenyl | 30 | 177-179 |
| 37 | 3-OCH₃-4-OH-phenyl | 25 | 170-172 |
| 38 | 1-naphtyl | 35 | 189-192 |

The present invention also describes the determination of the action mechanism of the synthesized acyl-hydrazones and their analogue and similar compounds comprising oxadiazole. The invention also refers to the use of all such compounds as prototype drugs, or drugs, for the treatment of leukemias, especially acute lymphoblastic leukemia (ALL), tumors and other diseases associated with cell proliferation such as inflammation.

The acyl-hydrazones described herein act selectively on leukemic cells with activity in nanomolar order when compared to its activity in healthy human lymphocytes, as will be described below in the form of an example. Shown here is the relevance of biological results, novel for the tested compounds.

In another embodiment, the present invention provides pharmaceutical compositions comprising the compounds described above in combination with excipients, carriers and pharmaceutically acceptable adjuvants.

As used herein, the use of the term “pharmaceutically acceptable” means a non-toxic, inert solid, liquid, semisolid excipient, diluent, auxiliary formulation of any type, or simply a sterile aqueous medium such as saline. Some examples of materials which can serve as pharmaceutically acceptable carriers are sugars such as lactose, glucose and sucrose, starches such as corn starch and potato starch, cellulose and its derivatives such as sodium carboxymethyl cellulose, ethyl cellulose and acetate cellulose, cyclodextrin; oils such as peanut oil, cottonseed oil, sunflower oil, sesame oil, olive oil, corn oil and soybean oil; glycols such as propylene glycol, polyols such as glycerin glycol, sorbitol, mannitol and polyethylene; esters such as ethyl laurate, ethyl oleate, agar; buffering agents such as aluminum hydroxide and magnesium hydroxide; alginic acid; pyrogen-free water; isotonic saline, Ringer's solution; ethyl alcohol and phosphate buffer solutions, as well as other non-toxic compatible substances used in pharmaceutical formulations.

Particularly, the pharmaceutical or veterinary compositions comprising the compounds of the present invention can be intended for administration by any type of administration, especially parenteral administration.

Specifically, the compositions of the present invention may comprise any type of excipient used in the production of drugs in any of the above pharmaceutical forms, such as absorbents, diluents, binders, disintegrants, lubricants, glidants, plasticizers, coating agents, matrix forming agents for controlled release, solvents and co-solvents, wetting agents, emulsifiers, surfactants, thickening agents, tonicity agents, humectants, levigating agents, agents to expel air, alkalizing or acidifying agents, preservatives, antioxidants, bactericides, bacteriostats, chelating agents, colorants and sweeteners.

Absorbents suitable for the compositions of the present invention may be any substance added to absorb the water present in the extracts or for setting certain volatile compounds, such as essences. Non-limiting examples of such excipients are calcium phosphate, kaolin, magnesium carbonate bentonite, and talc.

The compositions of the present invention may comprise, as solvent, any inert material added to formula to allow to obtain tablets or filling capsules with appropriate volumes and provide flow and compression properties necessary for production, for example, but not limited to, lactose, tribasic calcium phosphate, starch, mannitol, calcium sulfate, microcrystalline cellulose (Microcel, Avicel), dibasic calcium phosphate (Encompress, Ditab), magnesium oxide, magnesium carbonate, talc, kaolin, calcium carbonate, dextrose fructose, lactose, aspartame, cellulose, maltose, mannitol, guar gum, sorbitol, starch and sucrose.

Suitable binder substances for the compositions of this invention may be agents used to promote adhesion of the particles during granulation and compression of solid pharmaceutical forms, and can also be used in the compositions of the present invention, for example, carbopol, povidone, xanthan gum, gum arabic, alginic acid, compressible sugar, CMC-Na, ethylcellulose, gelatin, methylcellulose, povidone (PVP), starch, pregelatinized starch and liquid glucose in solution, dispersion or powder.

Disaggregants or disintegrants suitable for compositions of the present invention may be any component used for accelerating disintegration and/or dissolution of the pharmaceutical form in biological fluids, for example, alginic acid, starch, sodium alginate, CMC-Na, microcrystalline cellulose, croscarmellose sodium (Ac-Di-sol), sodium starch glycolate (Explotab) and crospovidone (Kollidon CL).

Lubricants suitable for compositions of this invention may be, for example, magnesium stearate, calcium stearate, stearic acid, talc and hydrogenated vegetable oil (e.g., Lubritab).

Glidants suitable for the compositions of the present invention may be, for example, colloidal silica (Aerosil 200) and talc.

Plasticizer agents suitable for the compositions of the present invention can be used with polymers to modify the phase transition temperature thereof and facilitate coalescence of formed films on granules, tablets or pellets. Non-limiting examples of such agents are glycerin, triethyl citrate, dibutyl phthalate, silicone, and PPG.

Coating agents used for coating compositions of the present invention in the form of tablets, granules, capsules or pellets may be, for example, cellulose acetate phthalate, ethylcellulose, gellan gum, maltodextrin, methacrylates, methylcellulose, microcrystalline cellulose, shellac, carrageenan gum, waxes, shellacs, gelatin, cellulose derivatives (methyl or ethyl cellulose, cellulose acetate phthalate, hydroxypropyl methyl cellulose, cellulose acetate), copolymers of acrylic and methacrylic esters (Eudragit of type L100, RS 30D, RS PM, S100, among others), polyvinyl alcohol (PVA) and polyvinyl acetate.

Matrix forming agents for the controlled release possibly employed in the compositions of the present invention, in order to obtain extended and/or controlled release of the active principle(s) may be HPMC, CMC-Na, xanthan gum, Carbopol, various types of Eudragit, agar-agar, polyoxyethylene derivatives (PEO's), cyclodextrin nanospheres and nanoparticles of any nature.

Solvents and co-solvents such as ethanol, corn oil, cottonseed oil, glycerin, isopropyl alcohol, mineral oil, oleic acid, peanut oil, purified water, water for injection, among others, can also be used in the compositions of the present invention.

Wetting agents suitable for the compositions of the present invention may be any substance added for the purpose of reducing the surface tension at the liquid/solid interface, for example, sodium lauryl sulfate (SLS), sodium docusate and polysorbates 20, 60, 80 (Tween 20, 60, 80).

Emulsifying agents suitable for the compositions of the present invention may be, for example, glyceryl monostearate, cetyl alcohol and gelatin and auxiliaries such as CMC-Na, MC, alginate and pectin.

Surfactant agents such as, for example, benzalkonium chloride, nonoxinol 10, octoxinol 9, polysorbate 80 and sodium lauryl sulfate are also adequate for the compositions of the present invention.

Thickening agents suitable for the compositions of the present invention may be any substance used to increase the consistency of ointments, for example, cetyl alcohol, white wax, yellow wax, stearyl alcohol, paraffin, microcrystalline wax, and cetyl esters wax.

Tonicity agents suitable for the compositions of the present invention may be any substance used to obtain solutions having osmotic characteristics similar to those of biological fluids to be administered by the ocular, nasal, parenteral routes such as NaCl (0.9%), mannitol (5.07%) and dextrose (5.51%).

Humectants suitable for compositions of the present invention can be glycerin, propylene glycol and sorbitol.

Levigating agents suitable for the compositions of this invention can be any liquid used as a facilitating agent in the process of reducing the drug particles during preparation of emulsions, oily bases, among others, for example, mineral oil (liquid petrolatum), glycerin, propylene glycol process, PEG 400, cottonseed oil, castor oil and polysorbate 80 (Tween® 80).

Agents for expelling air suitable to the compositions of this invention may be employed to expel the air from hermetically sealed enclosures or from fluid formulations to increase the stability, for example nitrogen (N₂) and carbon dioxide (CO₂).

Alkalizing or acidifying agents such as citric acid, ammonia, acetic acid, ammonium carbonate, fumaric acid, diethanolamine, hydrochloric acid (HCl), monoethanolamine, tartaric acid, potassium hydroxide (KOH), boric acid, sodium hydroxide (NaOH), sodium bicarbonate, sodium borate, and triethanolamine are also suitable for the compositions of the present invention.

Preservatives that can be used in the compositions of the present invention are, for example, antifungal agents such as benzoic acid, sodium benzoate, sodium butylparaben, methylparaben (Nipagin), propylparaben (Nipasol), ethylparaben, sodium propionate, and antibacterials such as benzalkonium chloride, benzethonium chloride, benzyl alcohol, cetylpyridinium, chlorobutanol and phenol chloride.

Antioxidants suitable for the compositions of the present invention may be selected from the group comprising butylated hydroxyanisole (BHA), butylated hydroxytoluene (BHT), β-tocopherol, ascorbic acid, ascorbyl palmitate, sodium metabisulfite, ethylenediamine tetraacetic acid (EDTA), citric acid, cysteine, glutathione vitamin C, sodium metabisulfite, cysteine and sodium thiosulfate.

The compositions of the present invention may further comprise, as buffering agents, citrate buffer, phosphate buffer and borate buffer. As colorants, flavors and flavorings, vanilla, menthol, cinnamon oil, anise oil and cocoa can be used, for example. Sweeteners may include, for example, aspartame, dextrose (glucose), mannitol, sorbitol, saccharin, sodium cyclamate, sugar, acesulfame potassium, stevioside and sucralose.

The compositions of the present invention may further comprise excipients such as bactericides, bacteriostats, antioxidants, preservatives, buffers, stabilizers, pH adjusters, osmolarity adjusters, antifoaming agents and surfactants, as well as residue of inactivation agents or fractionation antigens, components of growth media and solvents commonly used in the production of medicines. Examples of these types of components can be found in “The Handbook of Pharmaceutical Excipients” (RAYMOND C. Rowe, Publisher The Pharmaceutical Press).

A variety of routes of administration of the compositions described herein is available. The particular mode selected will depend on the particular active ingredient selected, the dosage required for therapeutic efficacy and the patient to which the composition is administered.

In another embodiment, the present invention describes the use of the compounds and compositions described herein for the treatment of diseases associated with cell proliferation, such as acute lymphoblastic leukemia (ALL), tumors and inflammation.

In another embodiment, the present invention provides methods of treating diseases associated with cell proliferation such as acute lymphoblastic leukemia (ALL), tumors and inflammation using the compounds and compositions described herein for the treatment of diseases associated with cell proliferation such as acute lymphoblastic leukemia (ALL), tumors and inflammation.

### EXAMPLES

The present invention will now be described by way of illustrative examples.

### Example 1

### General Procedure for the Preparation of 3,4,5-Trimethoxyphenyl-Hydrazones 01-29

For the synthesis of 3,4,5-trimetoxiphenyl-hidrazones 01-29, we used the methodology described by Troeberg and col. (Troeberg, L.; Chen, X.; Flaherty, T. M.; Morty, R. E.; Cheng, M.; Hua, H.; Springer, C.; McKerrow, J. H.; Kenyon, G. L.; Lonsdale-Eccles, J. D.; Coetzer, T. H. T.; Cohen, F. E. Chalcone, acyl hydrazide, and related amides kill cultured Trypanosoma brucei brucei. Molecular Medicine, 2000, 6, 660-669). In a 100 mL/1 mouth reaction flask the 3,4,5-trimethoxyphenyl-hydrazide is placed (2 mmol), prepared as described in Example 2, in an organic solvent: acetone, ethyl acetate, ethyl ether, ethanol, methanol (20 mL) and the appropriate aldehyde (2 mmol). The mixture was refluxed for 1 to 10 hours. Then, the solution was filtered and the solid recrystallized in an organic solvent.

### Example 2

### General Procedure for Obtaining 3,4,5-Trimethoxyphenyl-Hydrazide Used to Obtain 3,4,5-Trimethoxyphenyl-Hydrazones 01-29

For the synthesis of 3,4,5-trimethoxyphenyl-hydrazide used in the preparation of acyl-hydrazones 01-29, we used the methodology already described (Chida, A. S.; Vani, P. V. S. N.; Chandrasekharam, M.; Srinivasan, R.; Singh, A. K. Synthesis of 2,3-dimethoxy-5-methyl-1,4-benzoquinone: a key fragment in coenzyme-Q series. Synthetic communications, 31, 657-660, 2001), carried out in two steps:

Obtaining Ester:

In a 1000 mL/1 mouth reaction flask, gallic acid (50 g, 0.294 mol) was placed with dimethyl sulfate (178.1 g, 1.413 mol), anhydrous potassium carbonate (175.5 g, 1.293 mol) and TBAI (tetrabutylammonium iodide) (1 g) in an organic solvent which may be ethanol, ethyl ether, ethyl acetate, acetone, petroleum ether (375 ml) and refluxed for 1 to 12 hours. The solid obtained was filtered and washed with the same organic solvent (3×50 mL). The ester was obtained in the form of a cream-colored amorphous solid, with a 78% yield; mp: 84° C. (lit. mp 82-83° C.). NMR ¹H (CDCl₃): 1.60 (s, 3H, CH₃), 3.92 (s, 9H, OCH₃), 7.33 (s, 2H, Ar).

Obtaining Hydrazide:

In a 1000 mL/1 mouth reaction flask, the ester obtained in the first step was placed (48 g, 0.212 mol), with a solution of 99% hydrazine hydrate (N₂H₄.H₂O) (77.6 g, 1.54 mol) and an organic solvent which may be ethanol, ethyl acetate, dichloromethane, acetone, methanol (200 mL). The mixture was refluxed for 1 to 5 h and maintained overnight under magnetic stirring only at a temperature between 0 and 50° C. The solid obtained was filtered and recrystallized in methanol to obtain the 3,4,5-trimethoxyphenyl-hydrazide as white crystals with a yield of 85%; mp: 162-163° C. (lit. mp 168° C.). NMR ¹H (CDCl₃): 3.80 (s, 3H, OCH₃), 3.90 (s, 6H, OCH₃), 7.18 (s, 2H, Ar), 9.55 (NH).

### Example 3

### General Procedure for the Preparation of Phenyl-Hydrazones 30-35

For the synthesis of phenyl-hydrazones 30-35, we used the methodology described by Troeberg and col. (Troeberg, L.; Chen, X.; Flaherty, T. M.; Morty, R. E.; Cheng, M.; Hua, H.; Springer, C.; McKerrow, J. H.; Kenyon, G. L.; Lonsdale-Eccles, J. D.; Coetzer, T. H. T.; Cohen, F. E. Chalcone, acyl hydrazide, and related amides kill cultured Trypanosoma brucei brucei. Molecular Medicine, 2000, 6, 660-669), in the same way as described for obtaining the 3,4,5-trimetoxiphenyl-hidrazones.

### Example 4

### General Procedure for the Preparation of Oxadiazoles 36-38

For the synthesis of oxadiazoles 36-38, we used the methodology described by Jin and col. (Jin, L.; Chen, J.; Song, B.; Chen, Z.; Yang, S.; Li, Q.; Hu, D.; Xu, R. Bioorg Med Chem, 2006, 16, 5036-5040). In a 100 mL/1 mouth reaction flask, the corresponding 3,4,5-trimetoxiphenyl-hidrazone (1 mmol) and acetic anhydride (10 mL) were mixed. The mixture was refluxed for 1 to 10 hours and it was cooled afterwards with the addition of crushed ice and left overnight at a temperature between 0 and 60° C. to precipitate the product. The solid obtained was filtered, washed with water and recrystallized with organic solvent/water.

Infrared (IR) Spectral Data and Nuclear Magnetic Resonance (Nmr) of ¹H and ¹³C of the Novel Compounds

* ppm relative to TMS, Multiplicity (J in Hz). Solvent CDCl₃.

05—¹H NMR (DMSO-d₆) 3.73 (s, 3H, p-OCH₃), 3.86 (s, 6H, m-OCH₃), 3.89 (s, 3H, m-OCH₃), 7.22 (s, 2H, H2, H6), 7.34 (s, 1H, H6′), 7.60 (s, 1H, H2′), 8.31 (s, 1H, HC═N), 10.08 (1H, OH), 11.66 (s, 1H, NH). ¹³C NMR (DMSO-d₆) δ6.78 (m-OCH₃), 60.81 (p-OCH₃), 85.17 (C3′), 105.84 (C2, C6), 109.72 (C6′), 128.28 (C1), 129.31 (C1′), 130.75 (C2′), 141.03 (C4), 147.35 (C═N), 147.97 (C5′), 149.00 (C4′), 153.36 (C3, C5), 163.16 (C═O). IRₘₐₓ/cm⁻¹ 3382 (N—H), 1636, 1228 (C═O), 1565 (C═N), 1290, 1045 (C—O), 2999, 2839, 1585, 1490, 1334, 1137, 997 (Ar) (KBr).

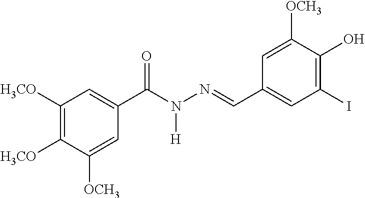 [SMILES: COc1cc(/C=N/NC(=O)c2cc(OC)c(OC)c(OC)c2)cc(I)c1O]

17—¹H NMR (DMSO-d₆) 3.74 (s, 3H, p-OCH₃), 3.88 (s, 6H, m-OCH₃), 7.23 (s, 2H, H2, H6), 7.53 (s, 1H, H2′), 7.66 (m, 1H, H4′), 8.02 (s, 1H, NH5′), 8.43 (s, 1H, HC═N), 11.37 (s, 1H, NH). ¹³C NMR (DMSO-d₆) δ6.76 (m-OCH₃), 60.81 (p-OCH₃), 105.71 (C2, C6), 129.51 (C1), 131.94 (C1′), 132.16 (C2′), 135.37 (C4′), 145.45 (C═N), 153.35 (C3, C5), 162.29 (C═O). IRₘₐₓ/cm⁻¹ 3212 (N—H), 1623, 1234 (C═O), 1580 (C═N), 1280, 1054 (C—O), 2994, 2941, 2838, 1503, 1456, 1411, 1344, 1125, 1006, 844 (Ar) (KBr).

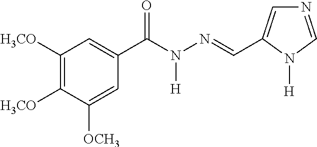 [SMILES: COc1cc(C(=O)N/N=C/c2cnc[nH]2)cc(OC)c1OC]

21—¹H NMR (DMSO-d₆) 3.71 (s, 3H, p-OCH₃), 3.79 (s, 6H, o-OCH₃), 3.85 (s, 6H, m-OCH₃), 6.72 (d, J=8.0 Hz, 2H, H3′, H5′), 7.23 (s, 2H, H2, H6), 7.34 (t, J=8.0 Hz, 1H, H4′), 8.60 (s, 1H, HC═N), 11.52 (s, 1H, NH). ¹³C NMR (DMSO-d₆) δ6.72 (m-OCH₃), 56.78 (o-OCH₃), 60.76 (p-OCH₃), 105.08 (C3′, C5′), 105.76 (C2, C6), 111.75 (C1′), 129.37 (C1), 131.87 (C4′), 140.88 (C4), 143.92 (C═N), 153.31 (C3, C5), 159.38 (C2′, C6′), 162.81 (C═O). IRₘₐₓ/cm⁻¹ 3186 (N—H), 1644, 1240 (C═O), 1586 (C═N), 1258, 1068 (C—O), 3002, 2928, 2838, 1502, 1473, 1417, 1378, 1342, 1121, 1007, 783 (Ar) (KBr).

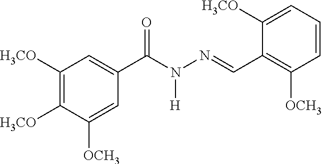 [SMILES: COc1cccc(OC)c1/C=N/NC(=O)c1cc(OC)c(OC)c(OC)c1]

26—¹H NMR (DMSO-d₆) 3.42 (s, 3H, p-OCH₃), 3.85 (s, 6H, m-OCH₃), 6.89 (d, J=8.6 Hz, 1H, H5′), 7.25 (s, 2H, H2, H6), 7.42 (d, J=8.6 Hz, 1H, H6′), 7.78 (s, 1H, H3′), 8.61 (s, 1H, HC═N), 11.26 (s, 1H, NH), 12.01 (1H, OH). IRₘₐₓ/cm⁻¹ 3220 (N—H), 1653, 1228 (C═O), 1588 (C═N), 1275, 1099 (C—O), 3004, 2941, 2834, 1550, 1503, 1479, 1463, 1416, 1352, 1335, 1189, 1011, 992, 951, 839, 760 (Ar) (KBr).

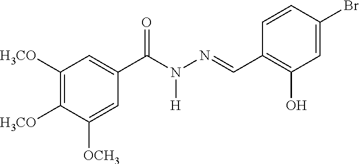 [SMILES: COc1cc(C(=O)N/N=C/c2ccc(Br)cc2O)cc(OC)c1OC]

29—¹H NMR (DMSO-d₆) 3.72 (s, 3H, p-OCH₃), 3.85 (s, 6H, m-OCH₃), 7.22 (s, 2H, H2, H6), 7.82 (d, J=8.6 Hz, 1H, H5′), 8.02 (d, J=8.6 Hz, 1H, H6′), 8.15 (s, 1H, H2′), 8.51 (s, 1H, HC═N), 11.96 (s, 1H, NH). ¹³C NMR (DMSO-d₆) 56.65 (m-OCH₃), 60.73 (p-OCH₃), 105.96 (C2, C6), 126.29 (C2′), 127.98 (C1′), 128.88 (C1), 132.31 (CF₃), 132.91 (C4′), 133.01 (C5′), 134.82 (C6′), 141.27 (C4), 145.61 (C═N), 153.40 (C3, C5), 163.50 (C═O). IRₘₐₓ/cm⁻¹ 3182 (N—H), 1655, 1242 (C═O), 1587 (C═N), 1269, 1039 (C—O), 3008, 2938, 2838, 1506, 1480, 1417, 1336, 1316, 1173, 1121, 1006, 958, 666 (Ar) (KBr).

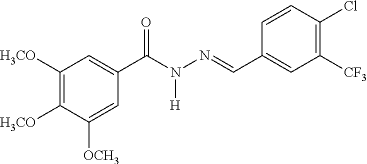 [SMILES: COc1cc(C(=O)N/N=C/c2ccc(Cl)c(C(F)(F)F)c2)cc(OC)c1OC]

36—¹H NMR (DMSO-d₆) 2.36 (s, 3H, CH₃), 3.85 (s, 3H, p-OCH₃), 3.91 (s, 3H, m-OCH₃), 3.92 (s, 6H, m-OCH₃), 6.00 (s, 1H, OH), 6.99 (s, 1H, H2′), 7.06 (s, 1H, H6′), 7.11 (s, 2H, H2, H6), 7.26 (s, 1H, HC—N). ¹³C NMR (DMSO-d₆) 21.75 (CH₃), 56.56 (m-OCH₃), 61.25 (p-OCH₃), 91.52 (C—N), 104.44 (C2, C6), 117.93 (C5′), 119.37 (C2′), 122.52 (C6′), 136.02 (C1′, C1), 139.24 (C4′), 141.49 (C4), 152.90 (C3′), 153.64 (C3, C5), 155.84 (C═N), 168.32 (C═O). IRₘₐₓ/cm⁻¹ 1766, 1238 (C═O), 1667, 1582 (C═N), 1254, 1047 (C—O), 1177 (C—N), 3445 (OH), 1130, 621 (C—Br), 3004, 2941, 2838, 1466, 1416, 1366, 1306, 1190, 998, 858 (Ar) (KBr).

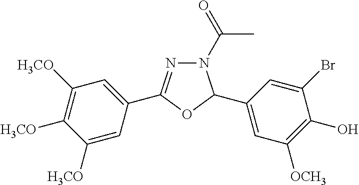 [SMILES: COc1cc(C2OC(c3cc(OC)c(OC)c(OC)c3)=NN2C(C)=O)cc(Br)c1O]

37—¹H NMR (DMSO-d₆) 2.35 (s, 3H, CH₃), 3.82 (s, 3H, p-OCH₃), 3.90 (s, 3H, m-OCH₃), 3.92 (s, 6H, m-OCH₃), 6.97 (s, 1H, H2′), 7.06 (m, 1H, H5′), 7.10 (s, 2H, H2, H6), 7.25 (s, 1H, HC—N), 7.44 (m, 1H, H6′); the signal corresponding to the OH group is not observed. ¹³C NMR (DMSO-d₆) 21.54 (CH₃), 56.27 (m-OCH₃), 56.33 (m-OCH₃), 61.02 (p-OCH₃), 91.14 (C—N), 104.22 (C2, C6), 109.77 (C2′), 119.16 (C5′), 128.09 (C6′), 136.402 (C1′, C1), 141.24 (C4′), 141.96 (C4), 151.78 (C3′), 153.42 (C3, C5), 155.60 (C═N), 168.10 (C═O). IR max/cm⁻¹ 1767, 1243 (C═O), 1665, 1581 (C═N), 1250, 1043 (C—O), 1177 (C—N), 3445 (OH), 1129, 644 (0-Br), 2967, 2945, 2838, 1507, 1466, 1417, 1365, 1036, 1287, 1197, 1083, 997, 958, 861, 699 (Ar) (KBr).

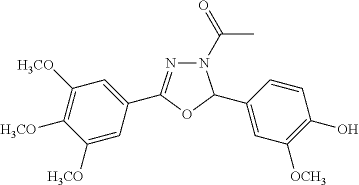 [SMILES: COc1cc(C2OC(c3cc(OC)c(OC)c(OC)c3)=NN2C(C)=O)ccc1O]

38—¹H NMR (DMSO-d₆) 2.47 (s, 3H, CH₃), 3.87 (s, 9H, OCH₃), 7.08 (s, 2H, H2, H6), 7.26 (s, 1H, HC—N), 7.26 (m, 1H, H2′), 7.48 (t, J=8.0 Hz, 1H, H3′), 7.55 (t, J=8.0 Hz, 1H, H7′, H8′), 7.62 (t, J=8.0 Hz, 1H, H7′), 7.76 (m, 1H, H4′), 7.92 (d, J=8.0 Hz, 1H, H6′), 8.22 (d, J=8.0 Hz, 1H, H9′). ¹³C NMR (DMSO-d₆) 21.57 (CH₃), 56.28 (m-OCH₃), 60.98 (p-OCH₃), 91.17 (C—N), 104.27 (C2, C6), 119.61 (C1), 123.04 (C2′), 125.07 (C9′), 125.20 (C3′), 126.05 (C7′), 126.95 (C8′), 128.95 (C4′), 130.51 (C6′), 130.61 (C10′), 130.79 (C5′), 134.04 (C1′), 141.11 (C4), 153.29 (C3, C5), 155.83 (C═N), 168.26 (C═O). IRₘₐₓ/cm⁻¹ 1731, 1243 (C═O), 1669, 1587 (C═N), 1254, 1039 (C—O), 1124 (C—N), 2997, 2941, 2827, 1509, 1465, 1416, 1369, 1332, 1191, 1006, 980, 847, 784, 699 (Ar) (KBr).

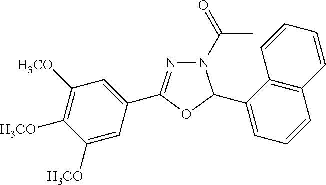 [SMILES: COc1cc(C2=NN(C(C)=O)C(c3cccc4ccccc34)O2)cc(OC)c1OC]

### Example 5

### Effect of Compounds on Acute Lymphoblastic Leukemia (all) Cell Lineages

a) Dilution and Storage of Compounds:

All compounds were resuspended in DMSO at stock concentration of 20 mM and stored at −20° C. For cytotoxicity testing, the dilutions from the stock solutions were made in culture medium (RPMI-1640 plus 10% of fetal bovine serum, 100 IU/ml penicillin and 100 pg/mL Streptomycin), immediately before being discharged to the cells.

b) Assays of In Vitro Sensitivity/Resistance of the Cells to the Compounds by the MTT Method:

The REH and Jurkat cell lines were maintained in

RPMI-1640 medium with 10% FBS (fetal bovine serum), 100 IU/mL penicillin, 100 pg/mL streptomycin and incubated at 37° C. and 5% CO₂. Cytotoxicity assays were performed according to the method described in the literature (Pieters, R.; et al. In vitro drug sensitivity of cells from children with leukemia using the MTT assay with improved culture conditions. Blood, 1990, 76, 2327-2336; Pieters, R.; et al. Relation of cellular drug resistance to long-term clinical outcome in childhood acute lymphoblastic leukaemia. Lancet, 1991, 338, 399-403 e Kaspers, G. J.; et al. In vitro cellular drug resistance and prognosis in newly diagnosed childhood acute lymphoblastic leukemia. Blood, 1997, 90, 2723). Cells were resuspended at a concentration of 3.75 10⁵ cells/mL in culture medium as described. Eighty microliters of this suspension were seeded into plates with 96 round-bottom wells, containing 20 microliters of different concentrations of compound or vehicle alone. Each treatment was done in triplicate. After 48 hours of incubation at 37° C. and 5% CO₂, 20 μL of MTT solution was added (5 mg/mL of 1x PBS), followed by further incubation for 4 hours and 30 minutes at 37° C. and 5% CO₂. During these 4 hours and 30 minutes, MTT (yellowish) is metabolized to formazan salt (bluish in color) by living cells. After this, 100 μL sodium dodecyl sulphate (SDS) 10%+0.1M of HCl were added to dissolve the formazan crystals. After incubating overnight, absorbance reading at 570 nm was carried out. The percentage of cells surviving the treatment were calculated relative to the number of surviving cells in the medium without addition of the compounds in question (“negative control”).

c) Determination of IC: IC₅₀ is defined as the concentration of a compound at which 50% of maximal inhibition is obtained. After reading the absorbance, survival curves were constructed and the values of IC₅₀ were obtained with the GraphPad Prism software.

### Results: Action of Acil Hydrazones and Oxadiazoles on B and T Lineage Leukemia Cells (HEK and Jurkat Respectively)

The cytotoxic effect of the synthesized compounds on human leukemia Jurkat and HEK cells was studied by cell viability assay (MTT) according to the methods described by Pieters et al (Pieters, R.; et al. In vitro drug sensitivity of cells from children with leukemia using the MTT assay with improved culture conditions. Blood, 1990, 76, 2327-2336; and Pieters, R.; et al. Relation of cellular drug resistance to long-term clinical outcome in childhood acute lymphoblastic leukaemia. Lancet, 1991, 338, 399-403) and Kaspers et al (Kaspers, G. J.; et al. In vitro cellular drug resistance and prognosis in newly diagnosed childhood acute lymphoblastic leukemia. Blood, 1997 90, 2723). The results are shown in Table 3.

TABLE 3
Screening of compounds 01-29 in 0.1 DM in human leukemic Jurkat
cells and HEK highlighting the most active compounds).

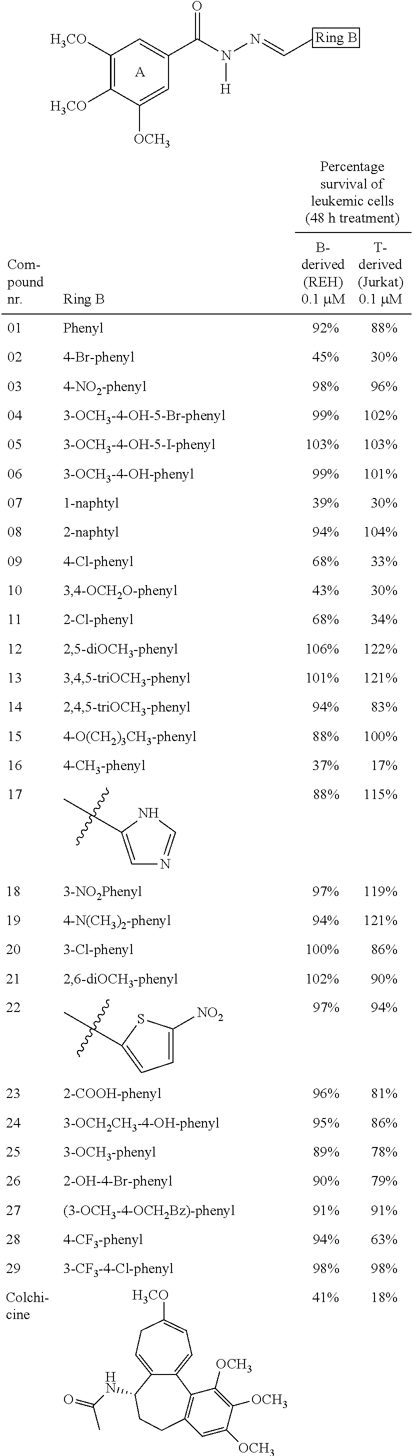

In order to analyze the influence of the methoxy groups in the A ring of acyl-hydrazones 01-29, we tested the series of complementary acyl-hydrazones (30-35). None of the compounds in this series showed activity against cells in the screening performed, thus proving the necessity of the trimethoxylated ring for antileukemic activity.

In a third attempt, the search for compounds correlated to the active ones, the cyclic derivatives 1,3,4-oxadiazoles (36-38) of 3,4,5-trimethoxyphenyl-hydrazones were tested. Similarly to the complementary acyl-hydrazones (30-35), none of the 1,3,4-oxadiazoles (36-38) was shown to be active. The explanation of these results may be due to the rigidity of the rings promoted by the cyclization, preventing the interaction of the molecule with the target.

Thus, the compounds 02, 07, 09, 10, 11 and 16 were selected to have their IC₅₀ determined; values are presented in Table 4.

TABLE 4
IC₅₀ of the most active compounds in leukemic Jurkat cells and REH.

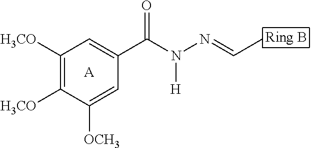

| | | IC₅₀ (nM) - | |
|---|---|---|---|
| | | 48 h of treatment | |
| | | B | T |
| Compound nr. | Ring B | (REH) | (Jurkat) |
| 02 | 4-Br-phenyl | 33.7 | 31.4 |
| 07 | 1-naphtyl | 25.4 | 15.7 |
| 09 | 4-Cl-phenyl | 328.3 | 202.9 |
| 10 | 3,4-OCH₂O-phenyl | 206.2 | 154.5 |
| 11 | 2-Cl-phenyl | 458.7 | 189.4 |
| 16 | 4-CH₃-phenyl | 70.6 | 71.5 |
| Colchicine | 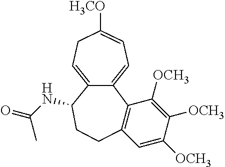 [SMILES: COC1=CC=C2C(=CC1)[C@@H](NC(C)=O)CCc1cc(OC)c(OC)c(OC)c12] | 9.5 | 7.7 |

The acyl-hydrazones 02 and 07 showed excellent results, with an IC₅₀ of 33.7 nM and 25.4 nM for the leukemic strain REH and 31.4 nM and 15.7 nM for Jurkat.

### Example 6

### Experiments for Determining the Action Mechanism of the Compounds with DNA Microarrays

The cells were incubated for 12 hours in fresh medium before the experiments. Two million cells from the leukemic cell line HL60, in triplicate, were treated with a 125 nM dose of compound 07 or with dimethylsulfoxide (DMSO) vehicle for 6 h, in DMEM supplemented with 10% fetal bovine serum. At the end of this period, the cells were recovered by brief centrifugation and lysed in a solution of guanidine of the RNAspin Mini RNA Isolation kit (GE).

a) Purification of RNA and Preparation of the Biotinylated Probe:

Total RNA was extracted from cells using the RNAspin Mini RNA Isolation kit (GE) following the manufacturer's instructions. A total of 100 ng of RNA was used to prepare biotinylated complementary RNA probes (Bio-cRNA), by cDNA synthesis followed by amplification by in vitro transcription, using the GeneChip WT cDNA Synthesis and Amplification kit (Affymetrix) according to the recommendations of the manufacturer.

b) Oligonucleotide Microarrays and Hybridization Arrays:

the cRNA probe of each replica was hybridized in an oligonucleotide microarray of the human genome GeneChip Human Gene 1.0 ST Array (Affymetrix). Hybridization and subsequent washes and development of microarrays were performed following manufacturer's recommendations.

c) Data Analysis:

the results of the microarray hybridization were read in the Affymetrix Gene Chip Scanner 3000-7G. Data were analyzed in the Bioconductor platform. To obtain the values of gene expression, we used the iterPLIER-F16 algorithm at the gene level with the Affymetrix Expression Console. Genes differentially expressed in response to compound 07 were obtained by regression analysis using the LIMMA package and the Fold Change (FC) criteria >1.5 and p<0.05. The list of differentially expressed genes were analyzed using the CMap (http://www.broad.mit.edu/cmap/) for identification of possible action mechanisms related to the action of known compounds (Lamb, J., Crawford E. D. The Connectivity Map: Using Gene-Expression Signatures to Connect Small Molecules, Genes and Disease Science 2006, 313, 1929-1935).

To establish whether a given gene set components (gene set) are co-regulated in the microarray experimental data, a Gene Set Expression Analysis (GSEA; http://www.broadinstitute.org/gsea/) was used. The GSEA algorithm creates a list of genes represented on the microarrays, arranged with differential expression between the two experimental groups (treated with compound 07 and control). In this case, the genes that are suppressed by compound 07 are at the top of the list, while genes that are induced are at the bottom. The algorithm will then find in this list the genes that make up a certain gene pool. If these genes are significantly over-represented at the top of this list, it can be said that the whole gene is repressed by the compound 07. In the converse situation, the whole gene is induced by drug treatment. The gene sets analyzed by GSEA were obtained from several public databases (BioCarta, Signaling Pathway Database, Signaling Gateway, Signal Transduction Knowledge Environment, Human Protein Reference Database, GenMAPP, KEGG, Gene Ontology, Sigma-Aldrich Pathways, Gene Arrays BioScience Corp., Human Cancer Genome Anatomy Consortium and NetAffx). The mean expression values of probe sets related to the same gene were considered, using 1000 permutations as the setting of the False Discovery Rate (FDR) q-value. Gene sets with less than 5 and more than 500 components were not considered.

Results: Action Mechanism Proposed by Studies with DNA Microarrays

In order to determine the action mechanism of these compounds, we selected compound 07 for analysis of gene expression through a DNA biochip. These trials have generated a dataset (algorithms) that were subsequently interpreted by Connectivity Maps (CMap) (www.broadinstitute.org/cmap) and GSEA analysis.

Treatment of HL-60 cells with compound 07 resulted in transcription repression of 102 genes and in the transcription activation of 353 genes (FC>1.5, p<0.05).

The results obtained by the analysis of microarray gene expression, of the cells treated with the test compound 07, were analyzed in CMap. Genes differentially expressed in cells under the action of compound 07 were cross linked with a series of gene lists responsive to different drugs. These lists are part of the CMap database. When taken together, the drug data grouped by the ATC system (Anatomical Therapeutic Chemical; http://www.whocc.no/atcddd/), the effect of compound 07 was most similar to the effect of the drugs of the PO2CA group, that are Benzimidazole derivatives (e.g., albendazole, mebendazole, fenbendazole, thiabendazole) used as anthelmintics. The results of the analysis of compound 07 in CMap are shown in Table 5a below.

The action mechanism of these drugs is through the inhibition of tubulin and have been recently described as potential candidates for the treatment of leukemias (Spagnuolo P A, et al, The antihelmintic flubendazole inhibits microtubule function through a mechanism distinct from Vinca alkaloids and displays preclinical activity in leukemia and myeloma. Blood 2010; 115(23):4824-33) and solid tumors (Doudican N, et al, Mebendazole induces apoptosis via Bcl-2 inactivation in chemoresistant melanoma cells. Mol Cancer Res 2008; 6(8):1308-315; Gupta K, et al; antifungal antimitotic compound benomyl inhibits brain microtubule polymerization and dynamics and cancer cell proliferation at mitosis, by binding to a novel site in tubulin. Biochemistry 2004; 43(21):6645-6655).

TABLE 5a
Compounds positively correlated with the compound 07 by ATC code.

| | ATC | | | Enrich- | | | Non- |
|---|---|---|---|---|---|---|---|
| Rank | Code | Average | n | ment | p | Specificity | null % |
| 1 | P02CA | 0.404 | 16 | 0.511 | 0.00018 | 0.017 | 68 |
| 2 | C01AA | 0.541 | 10 | 0.63 | 0.00022 | 0.1852 | 80 |
| 3 | N05AC | 0.32 | 24 | 0.42 | 0.00026 | 0.2477 | 62 |
| 4 | P01AX | 0.415 | 9 | 0.638 | 0.00048 | 0.0194 | 77 |
| 5 | V03AF | −0.532 | 5 | −0.774 | 0.00106 | 0.0153 | 80 |
| 6 | P02CX | 0.585 | 6 | 0.724 | 0.00117 | 0.0884 | 83 |
| 7 | D06BX | −0.386 | 5 | −0.768 | 0.00124 | 0 | 60 |
| 8 | G04BD | 0.354 | 8 | 0.597 | 0.00279 | 0 | 62 |
| 9 | S01GA | 0.257 | 20 | 0.373 | 0.0056 | 0.0148 | 60 |
| 10 | G04CB | −0.314 | 6 | −0.642 | 0.00612 | 0.1281 | 50 |
| 11 | P02CE | 0.53 | 4 | 0.746 | 0.0079 | 0 | 100 |
| 12 | S01EX | 0.567 | 3 | 0.84 | 0.00803 | 0 | 100 |
| 13 | P02BX | 0.513 | 4 | 0.733 | 0.00985 | 0 | 0 |
| 14 | G03CC | 0.352 | 17 | 0.38 | 0.01053 | 0.05 | 64 |
| 15 | P03AA | 0.47 | 5 | 0.666 | 0.01093 | 0.1214 | 80 |
| 16 | N05CE | −0.294 | 4 | −0.708 | 0.01486 | 0.0423 | 50 |
| 17 | R01AA | 0.248 | 16 | 0.376 | 0.01544 | 0.016 | 56 |
| 18 | R01AB | 0.248 | 16 | 0.376 | 0.01544 | 0.016 | 56 |
| 19 | D08AC | −0.455 | 5 | −0.639 | 0.01568 | 0.0376 | 80 |
| 20 | R02AA | −0.455 | 5 | −0.639 | 0.01568 | 0.0376 | 80 |

Compared to data of other perturbations that are part of the CMap platform, considering only data for the HL-60 cell line, the effect of compound 07 was most similar to the effect of tanespimycin (enrichment=0.699, p<0.001), which is a drug that inhibits Hsp90. Hsp90 is a chaperone that interacts and collocates with tubulin (C Garnier, et al, Heat-shock protein 90 (hsp90) binds in vitro to tubulin dimer and inhibits microtubule formation. Biochem Biophys Res Commun 1998, 250(2):414-9; Redmond T et al; Immunofluorescence colocalization of the 90-kDa heat-shock protein and microtubules in interphase and mitotic mammalian cells. Eur J Celi Biol 1989; 50(1):66-75; Sanchez E R et al; Evidence that the 90-kilodalton heat shock protein is associated with tubulin-containing complexes in L cell cytosol and in intact PtK cells. Mol Endocrinol 1988; 2(8):756-60; Czar M J et al; Immunofluorescence localization of the 90-kDa heat-shock protein to cytoskeleton Eur J Cell Biol 1996; 70 (4):322-30), protecting it from denaturation and keeping it in a compatible for polymerization (F Weis et al state, The 90-kDa heat tubulin shock protein Hsp90 protects against thermal denaturation. J Biol Chem 2010; 285(13):9525-34). The result suggests that, like tanespimycin, compound 07 can affect the process of assembling tubulin or destabilize the already formed microtubules.

TABLE 5b
Compound positively correlated with compound 07, for cell line.

| | | | | | | | Non- |
|---|---|---|---|---|---|---|---|
| Rank | ATC Code | Average | n | Enrichment | p | Specificity | null % |
| 1 | geldanamycin - MCF7 | 0.598 | 10 | 0.781 | 0 | 0.0168 | 90 |
| 2 | tanespimycin - MCF7 | 0.551 | 36 | 0.676 | 0 | 0.0263 | 80 |
| 3 | trichostatin A - MCF7 | −0.322 | 92 | −0.396 | 0 | 0.5347 | 55 |
| 4 | trichostatin A - PC3 | −0.275 | 55 | −0.385 | 0 | 0.5058 | 50 |
| 5 | tanespimycin - HL60 | 0.571 | 12 | 0.699 | 0.00002 | 0.0814 | 75 |
| 6 | methotrexate - HL60 | −0.835 | 2 | −0.989 | 0.00026 | 0 | 100 |
| 7 | helveticoside - PC3 | 0.8 | 2 | 0.987 | 0.00032 | 0.0128 | 100 |
| 8 | alvespimycin - MCF7 | 0.624 | 7 | 0.703 | 0.0005 | 0.0326 | 85 |
| 9 | 0175029-0000 - PC3 | 0.598 | 4 | 0.856 | 0.00056 | 0.0573 | 100 |
| 10 | PNU-0251126 - PC3 | −0.57 | 4 | −0.868 | 0.0006 | 0.0067 | 100 |
| 11 | puromycin - MCF7 | 0.763 | 2 | 0.98 | 0.0007 | 0.0699 | 0 |
| 12 | monorden - MCF7 | 0.414 | 12 | 0.534 | 0.0009 | 0.0909 | 58 |
| 13 | finasteride - MCF7 | −0.628 | 3 | −0.918 | 0.00096 | 0.019 | 100 |
| 14 | methyldopate - MCF7 | −0.726 | 2 | −0.978 | 0.00107 | 0.008 | 100 |
| 15 | albendazole - MCF7 | 0.765 | 2 | 0.964 | 0.00225 | 0 | 100 |
| 16 | PHA-00745360 - CF7 | −0.374 | 4 | −0.812 | 0.00229 | 0.0073 | 75 |
| 17 | bendroflumethiazide - | 0.636 | 3 | 0.894 | 0.00234 | 0 | 100 |
| | MCF7 | | | | | | |
| 18 | 0198306-0000 - | −0.688 | 2 | −0.964 | 0.00288 | 0 | 100 |
| | MCF7 | | | | | | |
| 19 | tanespimycin - PC3 | 0.41 | 12 | 0.496 | 0.00311 | 0.2159 | 66 |
| 20 | CP-690334-01 - PC3 | −0.431 | 4 | −0.799 | 0.00318 | 0.0362 | 75 |

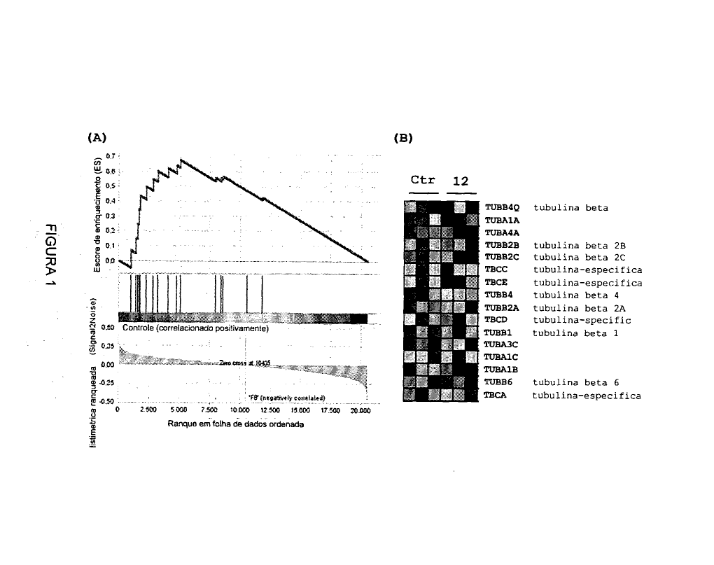

The dynamics of cellular processes demand continued cytoskeletal reorganization, therefore, the polymerisation and depolymerisation of tubulin. Tubulin inhibitors can act in two ways: (1) inhibiting tubulin polymerization or (2) stabilizing tubulin in such a way as to inhibit depolymerization. GSEA analysis showed that the compound 07 causes drastic reduction of the expression of a set of genes of tubulin and tubulin-specific chaperones (FIG. 1), which is typical of compounds which inhibit tubulin polymerization. Inhibition of tubulin polymerization results in accumulation of free tubulin (non polymerized) in the cell cytoplasm. The free cytoplasmic tubulin negatively autoregulates tubulin mRNA expression by suppressing the formation of new tubulin mRNA and accelerating the degradation of the existing mRNA (Caron J M, et al; autoregulation of tubulin synthesis in hepatocytes and fibroblasts. Celi J Biol 1985; 101:1763-72). In contrast, compounds which stabilize tubulin filaments lead to increased expression of tubulin.

### Example 7

### Effect of Compound 07 on the Progression of Cells Through the Cell Cycle, Activation of Regulators of the Cell Cycle and Induction of Apoptosis

The cells were incubated for 12 hours in fresh medium before experiments. Two million cells from the Jurkat line were incubated for 12 h, 18 h, 24 h and 30 h in RPMI-640 medium supplemented with 10% Fetal Bovine Serum (FCS) and 0.2% Penicillin/Streptomycin (PS) with a concentration of 125 nM of compound 07 or DMSO (vehicle).

a) Screening Assays of the Cell Cycle:

The cells treated or not with compound 07 were fixed in 70% ethanol for 2 hours, washed in PBS and incubated for 15 min at 37° C. in 1 mL of Triton X-100 0.1% 0.2 mg/mL of RNAse and 20 μg/mL propidium iodide in PBS. 20,000 events were collected on FACSCalibur flow cytometer with red fluorescence quantification, excluding any cell aggregates by the standard width vs. red fluorescence peak area. The data deconvolution to obtain the percentage of cells in each phase of cell cycle was performed using ModFit software v2.0.

B) Assays for Quantification of Apoptosis with Annexin V/Pi Labeling:

The cells treated or not with compound 07 were rinsed in PBS and labeled with Annexin V Apoptosis Assay kit (Invitrogen). Briefly, cells were resuspended in appropriate buffer containing calcium and incubated for 15 min with Annexin V-FITC and propidium iodide 5 μg/mL. 10,000 events were collected on FACSCalibur flow cytometer, excluding any debris by forward vs standard, side scatter, with quantification of green and red fluorescence.

c) Western Blot Testing for Analysis of Expression and Phosphorylation of Cell Cycle Regulatory Proteins.

The cells treated or not with compound 07 were lysed in buffer containing 50 mM Tris pH 7.7, 150 mM NaCl, 5 mM EDTA, 1% Sigma phosphatase inhibitor cocktail I, 1% Sigma phosphatase inhibitor cocktail II, 1% Sigma protease inhibitor cocktail II, 1% Igepal, 0.1% SDS, 0.5% sodium deoxycholate and 1 mM PMSF. After incubation for 30 min on ice and 3 cycles of freezing and thawing, extracts were centrifuged at 10,000 rpm for 20 min at 4° C. to sediment membrane debris. Of the supernatant, we proceeded with protein quantification by Bradford reagent (Biorad). The samples were boiled for 5 min with prior addition of β-mercaptoethanol and 100 μg of protein were subjected to acrylamide:bisacrylamide gel electrophoresis (39:1) 10% with SDS and electrotransferred to 0.45 μm nitrocellulose membrane (Schleicher & Schuell). Immunodetection was performed by incubation of the membrane with Cell Cycle/Checkpoint Antibody Sampler kit #9917 antibodies (Cell Signaling Technology) and Cyclin Dependent Kinase Inhibitor Antibody Sampler kit #9867 (Cell Signaling Technology) following the manufacturer's instructions. The development of the results was performed with the Super Signal West Pico Chemiluminiscent Substrate kit (Pierce) followed by exposure to X-ray film.

### Results: Compound 07 Induces Cell Cycle Arrest in G2 and Cell Death by Apoptosis

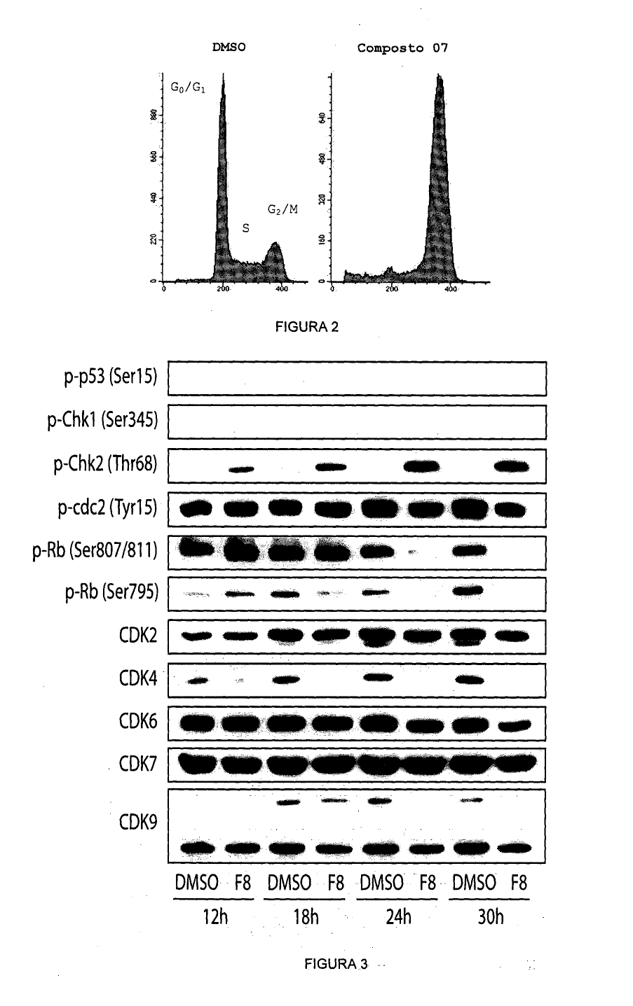

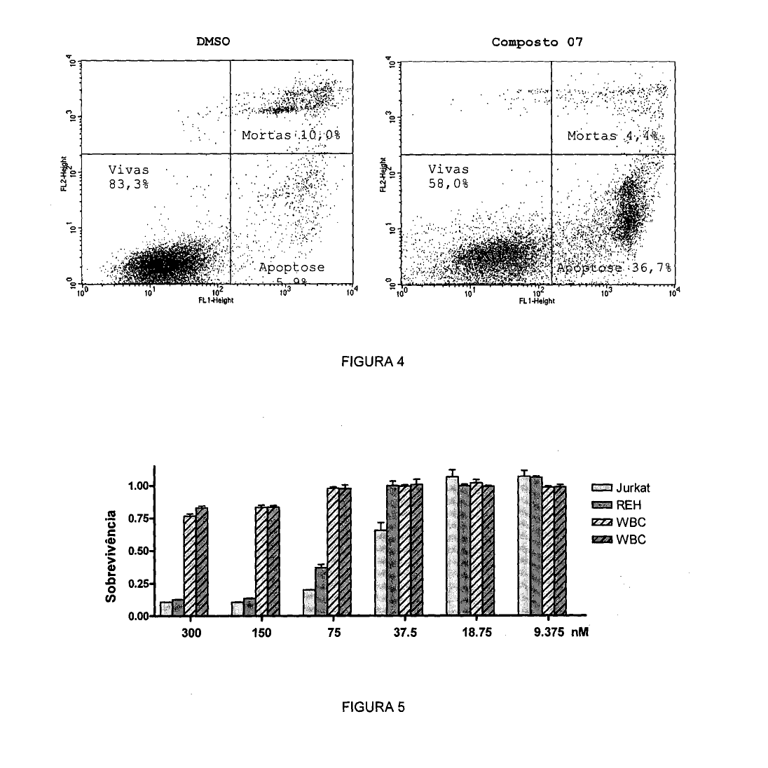

Results consistent with the inhibition of tubulin were obtained in the cell cycle analysis of Jurkat cells that were treated with compound 07. Jurkat cells treated with compound 07 showed cell cycle arrest in G2/M (FIG. 2).

Changes in the expression and phosphorylation state of various proteins that regulate the cell cycle were investigated. As shown in FIG. 3, after 12 hours of treatment with compound 07, it is possible to detect the phosphorylation of Chk2 in Thr68. Phosphorylation of the Thr68 residue is done by the Ataxia Telangiectasia Mutated factor (ATM) and results in activation of Chk2 and subsequent phosphorylation of a number of downstream targets, including cdc25, BRCA1, p53 and E2F, which are important control factors (chekpoints) of the cell cycle, repair of DNA damage and induction of apoptosis in response to DNA damage (Falck J, et al, The ATM-Chk2-Cdc25A checkpoint pathway guards against radioresistant DNA synthesis. Nature 2001, 410(6830): 842-7; Matsuoka S, et al; Linkage of ATM to cell cycle regulation by the Chk2 protein kinase. Science 1998, 282(5395): 1893-7; Lee J S et al; hCdsl-mediated phosphorylation of BRCA1 regulates the DNA damage response. Nature 2000: 404(6774) 201-4; Hirao A et al, DNA damage-induced activation of p53 by the checkpoint kinase Chk2. Science 2000, 287(5459): 1824-7; C Stevens et al, Chk2 activates E2F-1 in response to DNA damage. Nat Cell Biol 2003: 5(5): 401-9). The absence of detectable p53 results in the western blot (FIG. 3) can be explained by the low p53 expression in Jurkat cells (Vigorito E, et al;. Contributions of p53 and PMA to gamma-irradiation induced apoptosis in Jurkat cells. Hematol Cell Ther 1999, 41(4):153-61).

Treatment with Compound 07 resulted in the repression of the expression of CDK4 and to a lesser extent also of CDK6, shorter isoform of CDK2 and larger isoform of CDK9 (FIG. 3). D/CDK4/CDK6 cyclin and cyclin E/cdk2 complexes phosphorylate and inactivate Rb to allow cell cycle progression. The decrease in CDK2, CDK4 and CDK6 in cells treated with compound 07 decreases inactivation of Rb, which is then able to perform its duties of the inhibition of cell cycle progression and induce apoptosis.

### Example 8

### Effect of Compound 07 on Healthy Human Lymphocytes and on Colony Formation by Evaluating its Selectivity

a) In Vitro Assays of Sensitivity/Resistance of the Cells to the Compound 07 by the MTT Method:

for the test of compound 07 against normal lymphocytes, blood from human donors was separated in a ficoll gradient and the mononuclear cells were cultivated at 200,000 cells/well, in 80 microliters of RPMI-1640 (containing 10% FBS, 100 IU/mL penicillin and 100 μg/mL streptomycin) and 20 microliters of phytohemagglutinin solution (Cultilab) in each well for stimulation of T lymphocytes. Compound 07 was added in serial dilutions (50%) from 300 to 1 nm. As a control, parallel tests were run with cells from leukemic Jurkat and REH cell lines, and experiments with colchicine with the same concentrations as compound 07. Cytotoxicity was assessed after 48 hours by the MTT method, as described Above.

b) Colony formation experiments:

Cytotoxicity of compound 07 was also evaluated with a colony formation assay of hematopoietic cells. A “HSC-CFU complete with Epo” kit from Miltenyi (Cat #130-091-280) was used following the manufacturer's recommendations. Bone marrow cells from healthy donors were cultured in semi-solid methylcellulose medium supplemented with bovine fetal serum, bovine serum albumin, different growth factors (GM-CSF, G-CSF, SCF, IL-3, IL-6, Epo) and compound 07, whose action on hematopoiesis was evaluated. After two weeks of culture at 37° C. and 5% CO₂, an assessment of the number of granulocytes (CFU-G), macrophage (CFU-M), granulocyte/macrophage (CFU-GM), erythroid (BFU-E and CFU-E) and mixed (CFU-GEMM) colonies was carried out.

### Results: Selectivity of Compound 07

Compound 07 was tested on healthy and mature T lymphocytes stimulated with phytohemagglutinin to assess its action against normal cells. Colchicine was used as a positive control. The results are shown in Table 6.

TABLE 6
Percentage of survival in healthy and phytohemagglutinin-stimulated
lymphocytes treated with different concentrations of compound 07.

| Compound | Concentration (nM) | | | | | | | |
|---|---|---|---|---|---|---|---|---|
| nr. | 300 | 150 | 75 | 37.5 | 18.75 | 9.38 | 4.6 | 0 |
| 07 | 95% | 107% | 108% | 101% | 105% | 105% | 103% | 100% |
| Colchicine | 63% | 69% | 75% | 84% | 95% | 110% | 109% | 100% |

At concentrations of 300 nM compound 07 had a toxicity of 5% relative to the control, whereas colchicine at the same concentration caused 37% inhibition on the viability of lymphocytes T.

A dose-response analysis of compound 07 against leukemic cells as compared to normal lymphocytes stimulated with phytohemagglutinin (FIG. 5) shows that concentrations of up to 10 times higher than the cytotoxic dose for leukemic cells do not appear to affect normal lymphocytes induced by phytohemagglutinin. This indicates that compound 07 could act in leukemic cells without affecting normal immune function of patients.

To determine the effect of compound 07 in hematopoiesis, the same assay was tested on colony formation of bone marrow cells cultured in semi-solid methylcellulose medium plus growth factors. As shown in Table 7, compound 07 at concentrations very close to IC₅₀ (20 nM), displays inhibitory activity on erythrocyte formation comparable to a PI3K inhibitor, used in the assay as a positive control. Compound 07 also has an inhibitory activity against granulocytes and macrophages, although low in intensity when compared to a F13K inhibitor. At a 200 nM concentration, corresponding to 10 times the IC₅₀ for average LLA lineages, compound 07 completely inhibited hematopoiesis.

TABLE 7
Colony formation of bone marrow cells (%) using the compound 07.

| | | | | | | CFU- |
|---|---|---|---|---|---|---|
| | BFU-E | CFU-E | CFU-G | CFU-M | CFU-GM | GEMM |
| Negative control | 30.7 ± 13.9 | 27.0 ± 9.5 | 20.0 ± 4.0 | 40.7 ± 5.1 | 46.7 ± 14.6 | 5.7 ± 7.4 |
| 2 nM compound 7 | 39 | 23 | 11 | 17 | 52 | 1 |
| 20 nM compound 7 | 26 | 11 | 14 | 25 | 27 | 2 |
| 200 nM compound 7 | 0 | 0 | 0 | 0 | 0 | 0 |
| Pi3K inhibitor | 18 | 12 | 8 | 9 | 20 | 1 |

* For control the average ± standard deviation of 3 replicates are presented.

As reported above, five novel 3,4,5-trimethoxyphenyl-hydrazones and three novel 3,4-oxadiazoles were obtained. All acyl-hydrazones and oxadiazoles synthesized and included within the scope of the present invention were evaluated in leukemic cells of Jurkat and HEK strains, and the compounds 02 and 07 showed excellent antileukemic activity, similar to the standard compound (colchicine). The action mechanism of compound 07 was determined using DNA microarrays, showing its inhibitory activity of tubulin, with Chk2 activation and Rb, cell cycle arrest in G2/M and induction of cell death by apoptosis. Subsequent tests showed the selectivity of the compound 07 for leukemic cells in the order of 10 times, when compared to its action in healthy human lymphocytes.

The 3,4,5-trimethoxyphenyl-hydrazones and related compounds and their analogs, comprising the oxadiazoles, present in this invention, therefore have great potential as drugs prototypes, pre-drugs, or drugs, for treatment of different diseases associated with cell proliferation such as leukemias, including acute lymphoblastic leukemia (ALL), tumors and inflammation.

The description of the present invention has been presented for purposes of illustration, and detail for future applications. However, the present description has no limiting character regarding the inventions described and exemplified as disclosed herein. Changes and modifications compatible with the described above, and the skill or knowledge of the relevant art, are within the scope of the present invention. It's a legitimate intent that the invention comprises in its scope all modifications and variations of the same, according to the report description and claims.

1. Compound characterized by the structural formula I 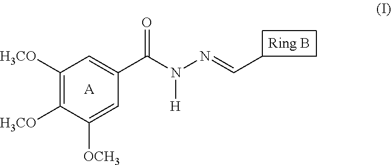
wherein Ring B is a substituent selected from the group comprising —NO₂-phenyl; 3-OCH₃-4-OH-5-Br-phenyl; 3-OCH₃-4-OH-5-I-phenyl; 2,4,5-triOCH₃-phenyl; CH₃-phenyl; 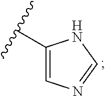 [SMILES: *c1cnc[nH]1]
3-NO₂-phenyl; 4-N(CH₃)₂-phenyl; 3-Cl-phenyl; 2,6-diOCH₃-phenyl; 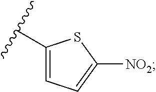 [SMILES: *c1ccc([N+](=O)[O-])s1]
2-COOH-phenyl; 3-OCH₂CH₃-4-0H-phenyl; 3-OCH₃-phenyl; 2-OH-4-Br-phenyl; (3-OCH₃-4-OCH₂phenyl)-phenyl; and 3-CF₃-4-Cl-phenyl.

2. Use of a compound characterized by the structural formula I, wherein the Ring B represents a substituent selected from the group comprising 1-phenyl, 4-Br-phenyl, 3-OCH₃-4-OH-5-I-phenyl 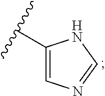 [SMILES: *c1cnc[nH]1]
1-naphtyl, 4-Cl-phenyl, 2,5-diOCH₃-phenyl; -3,4,5-triOCH₃-phenyl, 4-O(CH₂)₃CH₃-phenyl, —CF₃-phenyl, characterized in that it is for preparation of a medicament for treating cancer.

3. Compound characterized by the structure II 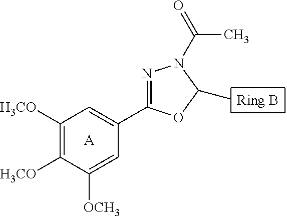
wherein Ring B represents a substituent selected from the group comprising 3-OCH₃-4-OH-5-Br-phenyl, 3-OCH₃-4-OH-phenyl and 1-naphthyl.

4. Pharmaceutical composition comprising one or more compounds described in any one of claims 1 to 3 and pharmaceutically acceptable excipients.

5. Use of the compound described in claim 1 or 3 characterized in that it is for the preparation of a medicament for treating diseases associated with cellular proliferation.

6. (canceled)

7. Use of the compound of formula Ia 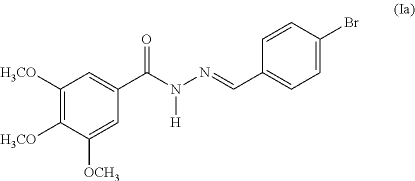 [SMILES: COc1cc(C(=O)N/N=C/c2ccc(Br)cc2)cc(OC)c1OC]
characterized in that it is for the preparation of a medicament for the treatment of diseases associated with cell proliferation.

8. Use of the compound of formula IIa 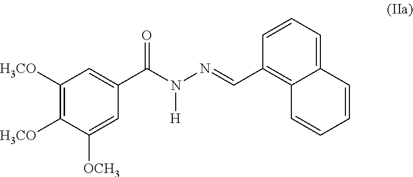 [SMILES: COc1cc(C(=O)N/N=C/c2cccc3ccccc23)cc(OC)c1OC]
characterized in that it is for the preparation of medicaments for the treatment of diseases associated with cell proliferation.

9. Use, according to any one of claims 7 and 8, wherein those diseases associated with cell proliferation are acute lymphoblastic leukemia (ALL), tumors and inflammation.

10. Method for treatment of diseases associated with cell proliferation comprising the administration of a compound described in any one of claims 1 to 3.
